(19) United States
(12) Patent Application Publication
(10) Pub. No.: US 2009/0209796 A1
(43) Pub. Date: Aug. 20, 2009

(54) Heteropolyacid salt catalyst, process for producing heteropolyacid salt catalyst and process for producing alkyl aromatic compound

(75) Inventor: Takuo Hibi, Ichihara-shi (JP)

Correspondence Address: WENDEROTH, LIND & PONACK, L.L.P., 1030 15th Street, N.W.,, Suite 400 East, Washington, DC, 20005-1503, US

(73) Assignee: SUMITOMO CHEMICAL COMPANY, LIMITED, Tokyo (JP)

(21) Appl. No.: 12/227,792

(22) PCT Filed: May 28, 2007
(86) PCT No.: PCT/JP2007/061232

§ 371 Date: Nov. 26, 2008

(30) Foreign Application Priority Data
May 29, 2006 (JP) 2006-147728
Apr. 27, 2007 (JP) 2007-118653

(51) Int. Cl. C07C 2/66; B01J 21/06

(52) U.S. Cl. 585/467; 502/254; 502/255; 502/200

(57) ABSTRACT

The present invention provides a heteropolyacid salt catalyst for use in an alkylation reaction of an aromatic compound or a transalkylation, disproportionation or isomerization reaction of an alkyl aromatic compound, which comprises a heteropolyacid salt catalyst represented by the following formula (1):

H₄₋ₘZₘSiXₗ₂O₄₀ (1)

wherein X represents W or Mo, Z represents (NH₄) or an alkali metal atom, and m represents a numerical value of 0≦m≦4, and comprising a heteropolyacid salt crystal having an average particle diameter in the short axis of the crystal of less than 300 nm as a main component, wherein said heteropolyacid salt catalyst has an acid amount on the external surface of not less than 190 μmol per weight of a heteropolyacid salt.

### TECHNICAL FIELD

The present invention relates to a heteropolyacid salt catalyst having particular structure, a process for producing the catalyst, and a process for producing an alkyl aromatic compound by alkylation of an aromatic compound, or transalkylation, disproportionation or isomerization of an alkyl aromatic compound using the catalyst. More particularly, the present invention relates to a heteropolyacid salt catalyst which poorly leaches out and provides high activity in the process for producing an alkyl aromatic compound, a process for producing the catalyst, and a process for producing an alkyl aromatic compound using the catalyst.

### BACKGROUND ART

There are many known heteropolyacid catalysts including a 12-tungstosilicic acid salt and a silica-supported 12-tungstosilicic acid catalyst, as described in JP-A 04-288026 and JP-A 05-025062. However, the known catalysts easily leach out or have lower activity, and thus their performance is insufficient. Therefore, there is a demand for development of a novel catalyst free from leaching out and having higher activity.

As a process for producing a heteropolyacid salt catalyst, JP-A 04-288026 and JP-A 05-025062 only disclose a production method of a 12-tungustosilicic acid salt using an aqueous solution under normal conditions. Heteropolyacid salt catalysts produced by the above method have lower activity and thus are insufficient. Therefore, there is a demand for development of a novel catalyst free from leaching but and having higher activity.

As a process for producing an alkyl aromatic compound by alkylation of an aromatic compound with olefin, for example, JP-A 04-288026 and JP-A 05-025062 disclose a method using a heteropolyacid salt catalyst. However, there is a problem that usual heteropolyacid salt catalysts have lower activity.

For a process for producing an alkyl aromatic compound by transalkylation or disproportionation comprising a reaction of an aromatic compound and/or an alkyl aromatic compound with a polyalkyl aromatic compound, usual supported heteropolyacid catalysts as described in JP-A 10-508300 have a problem that they leach out or have lower activity.

For a process for producing an alkyl aromatic compound by an isomerization reaction, usual supported heteropolyacid catalysts have a problem that they leach out or have lower activity.

### DISCLOSURE OF INVENTION

Under such circumstances, an object to be solved by the present invention is to develop a novel catalyst which can be used in producing an alkyl aromatic compound, and as a result, to provide a high-performance catalyst which poorly leaches out and has higher activity.

Another object of the present invention is to develop a process for producing a novel catalyst which can be used in producing an alkyl aromatic compound, and then to provide the novel catalyst to, in particular, a process for producing an alkyl aromatic compound by alkylation, transalkylation, disproportionation or isomerization.

Conventional catalysts leach out during a reaction or have insufficient activity. Therefore, a further object of the present invention is to provide a process for producing an alkyl aromatic compound by alkylation, transalkylation, disproportionation or isomerization using a novel catalyst which has high activity and does not leach out.

That is, the first aspect of the present invention relates to a heteropolyacid salt catalyst for use in an alkylation reaction of an aromatic compound or a transalkylation, disproportionation or isomerization reaction of an alkyl aromatic compound, which comprises a heteropolyacid salt catalyst represented by the following formula (1):

H₄₋ₘZₘSiXₗ₂O₄₀ (1)

wherein X represents W or Mo, Z represents (NH₄) or an alkali metal atom, and m represents a numerical value of 0≦m≦4, and comprising a heteropolyacid salt crystal having an average particle diameter in the short axis of the crystal of less than 300 nm as a main component, wherein said heteropolyacid salt catalyst has an acid amount on the external surface of not less than 190 μmol per weight of a heteropolyacid salt.

The second aspect of the present invention relates to a process for producing a heteropolyacid salt catalyst represented by the formula (1), which comprises preparing a heteropolyacid represented by the following formula (2) or the heteropolyacid supported on a carrier by salt formation in the presence of aliphatic alcohols or aliphatic alcohols containing an organic solvent and/or a water solvent from a solution of an ammonium or alkali metal compound, wherein X represents W or Mo.

H₄SiX₁₂O₄₀ (2)

The third aspect of the present invention relates to a process for producing an alkyl aromatic compound by alkylation, which comprises contacting an aromatic compound with olefin in the presence of the above described catalyst.

The fourth aspect of the present invention relates to a process for producing an alkyl aromatic compound by a transalkylation reaction or a disproportionation reaction, which comprises contacting an aromatic compound and/or an alkyl aromatic compound with a polyalkyl aromatic compound in the presence of the above described catalyst.

The fifth aspect of the present invention relates to a process for producing a di or more-substituted alkyl aromatic compound, which comprises performing an isomerization reaction for substitution positions of alkyl groups of a di or more-substituted alkyl aromatic compound in the presence of the above described catalyst.

According to the present invention, a novel catalyst which can be used in producing an alkyl aromatic compound can be developed, and as a result, a high-performance catalyst which poorly leaches out and has higher activity can be provided.

According to the present invention, a process for producing a novel catalyst which can be used in producing an alkyl aromatic compound can be also developed and provided.

Further, the developed novel catalyst can be provided to, in particular, a process for producing an alkyl aromatic compound by alkylation, transalkylation, disproportionation or isomerization.

### BRIEF DESCRIPTION OF DRAWINGS

FIG. 1 is a SEM image of a catalyst prepared in Example 1.

FIG. 2 is a dark field FE-STEM image of a catalyst prepared in Example 1.

FIG. 3 is a SEM image of a catalyst prepared in Example 2.

FIG. 4 is a dark field FE-STEM image of a catalyst prepared in Example 2.

FIG. 5 is a SEM image of a catalyst prepared in Reference Example 1.

FIG. 6 is a SEM image of a larger particle of a catalyst prepared in Reference Example 1.

FIG. 7 is a SEM image of a heteropolyacid salt prepared in Comparative Example 2.

FIG. 8 shows EDX spectra of a catalyst prepared in Example 1.

FIG. 9 shows EDX spectra of a catalyst prepared in Example 2.

FIG. 10 shows EDX spectra of a catalyst prepared in Reference Example 1.

FIG. 11 shows EDX spectra of a larger particle of a catalyst prepared in Reference Example 1.

### MODE FOR CARRYING OUT THE INVENTION

As heteropolyacid salt catalysts, many alkali metal salt of heteropolyacid including alkali metal partial salts of silicon-containing heteropolyacid have been conventionally proposed. However, unlike alkali metal partial salts of phosphorus-containing heteropolyacid, conventionally known alkali metal partial salts of silicon-containing heteropolyacid consist of large crystal particles and have insufficient catalytic activity. Particularly, the cesium partial salt of silicon-containing heteropolyacid, which is prepared by precipitate formation from an aqueous solution of a heteropolyacid containing silicon are extremely large crystals, and therefore it cannot exhibit sufficient activity. In this present invention, the word of “crystal” means polycrystal but not single crystal.

To the contrary, the heteropolyacid salt catalyst of the present invention is a novel heteropolyacid salt catalyst characterized in that the average particle diameter of the crystal is less than 300 nm. Therefore the heteropolyacid salt catalyst of the present invention exhibits extremely high activity because the crystal size is very small.

Hereinafter, a novel heteropolyacid salt catalyst which is the first aspect of the present invention will be explained.

A heteropolyacid used as a starting material is a heteropolyacid of the formula (2):

H₄SiX₁₂O₄₀ (2)

wherein X represents W or Mo.

That is, a heteropolyacid used as a starting material is 12-tungstosilicic acid or 12-molybdosilicic acid.

A counter cation is either (NH₄) or an alkali metal atom. Examples of a raw material for the counter cation include compounds such as ammonium, and carbonate, hydroxide and nitrate of an alkali metal.

The heteropolyacid salt catalyst of the present invention comprises a heteropolyacid salt crystal represented by the following formula (1):

H₄₋ₘZₘSiXₗ₂O₄₀ (1)

wherein X represents W or Mo, Z represents (NH₄) or an alkali metal atom, and m represents a numerical value of 0≦m≦4, as a main component, wherein the average particle diameter in the short axis of the crystal is less than 300 nm when the shape of said heteropolyacid salt is regarded as an elliptical shape, and is used for an alkylation reaction of an aromatic compound, or a transalkylation, disproportionation or isomerization reaction of an alkyl aromatic compound.

When the shape of the heteropolyacid salt is regarded as an elliptical shape, the average particle diameter in the short axis of the crystal is preferably not less than 1 nm and not more than 200 nm, more preferably not more than 150 nm, further more preferably not more than 100 nm, still more preferably not less than 1 nm and not more than 80 nm.

The average particle diameter is determined as follows.

The crystal particles of a heteropolyacid salt catalyst represented by the formula (1) are observed with an electron microscope. Some microscopic field photographs are randomly selected. Each of particles on the photographs is regarded as an elliptical shape, the diameter in the short axis of the crystal is measured, and the arithmetic average of the diameters is then obtained as an average particle diameter.

As an electron microscope, a scanning electron microscope and a transmission electron microscope are known. Relatively large crystal particles of not less than 50 nm can be observed with a scanning electron microscope.

In the heteropolyacid salt catalyst of the present invention comprising a heteropolyacid salt crystal having an average particle diameter in the short axis of less than 300 nm as a main component, the amount of heteropolyacid salt crystal particles having an average particle diameter in the short axis of less than 300 nm is preferably not less than 60% by weight, more preferably not less than 70% by weight, still more preferably not less than 80% by weight, most preferably not less than 90% by weight of the total heteropolyacid salt crystal particles.

The proportion of the heteropolyacid salt crystal particles is determined as follows. The crystal particles of the heteropolyacid salt catalyst are observed with an electron microscope. Some microscopic field photographs are randomly selected. The diameter in the short axis of each of crystals on the photographs is measured, and the particles are then divided into a group of an average particle diameter of less than 300 nm and a group of an average particle diameter of not less than 300 nm. It is preferable that the particle number in a group of an average particle diameter of less than 300 nm is not less than 60% of the total particle number.

The ratio of (NH₄) or an alkali metal atom to a Si atom, which is a value of m in the formula (1), is more than 0 and less than 4, preferably not less than 0.5 and less than 3.

Preferable examples of the heteropolyacid salt include an ammonium salt and an alkali metal salt of 12-tungstosilicic acid, further preferably a cesium salt of 12-tungustosilicic acid because a compound containing tungsten has more stronger acid.

The catalyst of the present invention has an acid amount on the external surface of not less than 190 μmol per weight of a heteropolyacid salt.

When a salt of 12-tungstosilicic acid or 12-molybdosilicic acid with an alkali metal is formed in aqueous solutions, the resulting salt becomes a large crystal particle and thus has a property of hardly exhibiting high catalytic activity. It is possible to prepare a catalyst having high activity by decreasing the crystal particle diameter to less than 300 nm. However, depending on a process for producing a catalyst, a catalyst having low activity may be obtained although it has a small crystal particle diameter. Therefore, in order to prepare a catalyst having high activity, it is important to increase the acid amount on the external surface of a catalyst as much as possible.

The acid amount of a catalyst is based on two kinds of acids, that is, an acid present in micropores of a catalyst and an acid present on the external surface of a catalyst. Since an acid site contributing to a Friedel-Crafts reaction such as alkylation of an aromatic compound is an acid site on the external surface, it is necessary to prepare a catalyst having a large acid amount on the external surface.

The acid amount on the external surface can be measured by a variety of methods. In the present invention, a temperature-programmed desorption method using 2,6-dimethylpyridine (hereinafter, abbreviated as 2,6-DMPy) is adopted. Although this measurement method cannot measure the correct acid amount completely on the external surface, it represents the acid amount on the external surface because the diffusion of 2,6-DMPy into micropores is difficult due to steric hindrance of 2,6-DMPy as compared with a temperature-programmed desorption method using pyridine (hereinafter, abbreviated as Py). The measurement method comprises calcining a catalyst under nitrogen and then adsorbing 2,6-DMPy on the catalyst. The acid amount of a catalyst is obtained by subtracting a decrease in the weight of the catalyst on which 2,6-DMPy is not adsorbed resulting from desorption during 300° C. to 900° C. (e.g. the amount of H₂O desorbed from the carrier, and the like) from a decrease in the weight of the catalyst on which 2,6-DMPy is adsorbed resulting from deporption during 300° C. to 900° C., and then dividing the obtained value by the molecular weight of 2,6-DMPy. Since the measurement is based on a decrease in weight and the all decreases are presumed to be due to 2,6-DMPy, it lacks precision. However, as the acid amount of a catalyst in the present invention, a numerical value obtained by this method is used.

The acid amount of the catalyst of the present invention is not less than 190 μmol/g-heteropolyacid salt (hereinafter, abbreviated as HPA), preferably not less than 190 μmol/g-HPA and not more than 1000 μmol/g-HPA, more preferably not less than 280 μmol/g-HPA and not more than 1000 μmol/g-HPA, still more preferably not less than 300 μmol/g-HPA and not more than 1000 μmol/g-HPA, most preferably not less than 400 μmol/g-HPA and not more than 1000 μmol/g-HPA. A detailed measurement method is shown in Examples.

Then, a process for producing a heteropolyacid salt catalyst which is the second aspect of the present invention will be explained.

A heteropolyacid used in preparation of the heteropolyacid salt represented by the formula (1):

H₄₋ₘZₘSiXₗ₂O₄₀ (1)

wherein X represents W or Mo, Z represents (NH₄) or an alkali metal atom, and m represents a numerical value of 0≦m≦4, is 12-tungstosilicic acid or 12-molybdosilicic acid represented by the formula (2):

H₄SiX₁₂O₄₀ (2)

wherein X represents W or Mo.

According to a conventional method of precipitate formation using an aqueous solution of a heteropolyacid and an aqueous solution of an ammonium or alkali metal compound, an extremely large crystal particle of a heteropolyacid salt was formed and it was difficult to obtain a micro crystallite. Thus, a process for producing a heteropolyacid salt catalyst comprising a heteropolyacid salt catalyst represented by the formula (1) and comprising a heteropolyacid salt crystal having an average particle diameter in the short axis of the crystal of less than 300 nm as a main component is the following method.

That is, it is a process for preparing a heteropolyacid represented by the formula (2) or the heteropolyacid supported on a carrier by salt formation in the presence of aliphatic alcohols solvent or aliphatic alcohols containing an organic solvent and/or a water solvent from a solution of an ammonium or alkali metal compound.

In addition to the aforementioned method, there are many examples of a process for producing a heteropolyacid salt catalyst comprising a heteropolyacid salt catalyst represented by the formula (1) and comprising a heteropolyacid salt crystal having an average particle diameter in the short axis of the crystal of less than 300 nm as a main component, and any process can be used in the present invention.

A specific example of the process will be explained below, but the process is not limited to the following described process. For example, a solvent for dissolving a heteropolyacid of the formula (2) is an aliphatic alcohol, aliphatic alcohols containing an organic solvent and/or a water solvent. As the aliphatic alcohol, an aliphatic alcohol having not more than 4 carbon atoms is preferably used, and examples thereof include various alcohols such as monoalcohols such as methanol, ethanol, propanol, isopropanol and butanol, diols such as ethylene glycol and propylene glycol, and glycerin. Among these, ethanol is preferably used. As an alcohol to be added to an aqueous solution, a water-soluble alcohol is preferably used, and examples thereof include various alcohols such as methanol, ethanol, isopropanol, ethylene glycol, propylene glycol, and glycerin. Among these, ethanol is preferably used.

A mixture of an aliphatic alcohol with an organic solvent is preferably used. Examples of the organic solvent include saturated aliphatic hydrocarbon, aromatic hydrocarbon, and ether, specifically, hexane, cyclohexane, heptane, benzene, and diethyl ether. More preferable examples include hexane and heptane.

Examples of an ammonium or alkali metal compound include ammonium, and carbonate, hydroxide and nitrate of an alkali metal, for example, ammonia water, and solutions of carbonate, hydroxide and nitrate of potassium, rubidium, cesium and the like. A compound which forms a salt when added to a solution of a heteropolyacid is preferably used. Among these, a solution of a cesium compound is preferably used.

The ratio of (NH₄) or an alkali metal atom to a Si atom, which is a value of m in the formula (1), is more than 0 and less than 4, preferably not less than 0.5 and less than 3.

Addition of a solution of an ammonium or alkali metal compound to a solution of a heteropolyacid is usually accomplished by adding dropwise a solution of an ammonia or alkali metal compound to a solution of a heteropolyacid under stirring. The temperature of a solution of a heteropolyacid is usually not lower than room temperature and not higher than the boiling point of a solvent. The addition is usually carried out under an atmospheric pressure. It is desirable that stirring is sufficiently performed.

Conversely, addition of a solution of a heteropolyacid or a supported heteropolyacid to a solution of an ammonium or alkali metal compound is usually accomplished by adding a solution of a heteropolyacid or a supported heteropolyacid to a solution of an ammonium or alkali metal compound under stirring. The temperature of a solution of an ammonium or alkali metal compound is usually not lower than room temperature and not higher than the boiling point of a solvent. The addition is usually carried out under an atmospheric pressure. It is desirable that stirring is sufficiently performed.

As a method for isolating a precipitate from a suspension of a heteropolyacid salt prepared as described above, there are many methods. Preferably, an evaporation to dryness method is used. The evaporation to dryness method includes preferably a method comprising heating a suspension to distill off a solvent, a method comprising isolating a precipitate using a rotary evaporator, and the like.

The temperature for evaporation to dryness is usually 30° C. to 100° C.

It is also preferable that a heteropolyacid is supported on a carrier, followed by formation of a salt with an ammonium or alkali metal compound. Examples of the carrier for supporting a heteropolyacid include silica, titanium oxide, zirconium oxide, activated carbon, alumina, niobium oxide, magnesium oxide, vanadium oxide, manganese oxide, iron oxide, tantalum oxide, and the like, preferably silica, titanium oxide, zirconium oxide and activated carbon, further preferably silica and activated carbon.

An example of a method for supporting a heteropolyacid on a carrier comprises mixing a heteropolyacid and a carrier powder in the presence of a solvent to obtain a suspension and then distilling off the solvent from the suspension with stirring. Examples of the solvent include aliphatic alcohols exemplified above for preparation of a heteropolyacid salt, water, and the like. A method for supporting a heteropolyacid on a carrier is not limited to the above method, and any method may be used as long as it is a method of supporting a heteropolyacid on a carrier.

Further, a heteropolyacid salt catalyst may be used in the form of being supported on a carrier. Examples of the carrier for supporting the catalyst include silica, titanium oxide, zirconium oxide, activated carbon, alumina, niobium oxide, magnesium oxide, vanadium oxide, manganese oxide, iron oxide, tantalum oxide, and the like, preferably silica, titanium oxide, zirconium oxide and activated carbon, further preferably silica and activated carbon.

An example of a method for supporting the catalyst on a carrier comprises mixing a heteropolyacid salt which has been evaporated to dryness and a carrier powder in the presence of a solvent to obtain a suspension, and then distilling off the solvent from the suspension with stirring. Examples of the solvent include aliphatic alcohols exemplified above for preparation of a heteropolyacid salt, water, and the like. Another example of a method for supporting the catalyst on a carrier comprises suspending a carrier powder in a solvent in advance, and then the catalyst is supported on the carrier while adding a solution of an ammonium or alkali metal compound to a solution of a heteropolyacid in the presence of the solvent to form a precipitate. A method for supporting a heteropolyacid salt catalyst on a carrier is not limited to the above methods, and any method may be used as long as it is a method of supporting a heteropolyacid salt on a carrier.

When a carrier is used, the ratio by mass of a heteropolyacid salt to the carrier is usually 1:0.1 to 1:100.

The temperature for evaporation to dryness is usually 30° C. to 100° C.

Specific examples of a method for preparing a heteropolyacid salt catalyst include various methods. A preferable example of a method for preparing a heteropolyacid salt catalyst comprises

a heteropolyacid dissolving step: which comprises dissolving a heteropolyacid represented by the following formula (2):

H₄SiX₁₂O₄₀ (2)

wherein X represents W or Mo, in a saturated aliphatic hydrocarbon organic solvent containing aliphatic alcohols;

an alkali solution preparation step: which comprises dissolving an alkali metal compound in an aliphatic alcohol;

a heteropolyacid salt formation step: which comprises adding a solution prepared in the alkali solution preparation step to a heteropolyacid solution prepared in the heteropolyacid dissolving step to form a heteropolyacid salt; and

a solvent evaporation step: which comprises evaporating a solvent from a mixture of a suspension containing aliphatic alcohols and a heteropolyacid salt catalyst prepared in the heteropolyacid salt formation step to isolate the catalyst as a solid.

A more preferable example of a method for preparing a heteropolyacid salt catalyst comprises

a heteropolyacid support step: which comprises supporting a heteropolyacid represented by the following formula (2):

H₄SiX₁₂O₄₀ (2)

wherein X represents W or Mo, on a carrier to prepare a supported heteropolyacid;

an alkali solution preparation step: which comprises dissolving an ammonium or alkali metal compound in an aliphatic alcohol solvent or aliphatic alcohols containing an organic solvent and/or a water solvent;

a heteropolyacid salt formation step: which comprises adding the supported heteropolyacid to a solution prepared in the alkali solution preparation step to form a heteropolyacid salt; and

a solvent evaporation step: which comprises evaporating a solvent from a mixture of a suspension containing aliphatic alcohols and a heteropolyacid salt catalyst prepared in the heteropolyacid salt formation step to isolate the catalyst as a solid.

A still more preferable example of a method for preparing a heteropolyacid salt catalyst comprises

a heteropolyacid support step: which comprises

supporting a heteropolyacid represented by the following formula (2):

H₄SiX₁₂O₄₀ (2)

wherein X represents W or Mo, on a carrier to prepare a supported heteropolyacid;

an alkali solution preparation step: which comprises dissolving an alkali metal compound in a saturated aliphatic hydrocarbon organic solvent containing aliphatic alcohols;

a heteropolyacid salt formation step: which comprises adding the supported heteropolyacid to a solution prepared in the alkali solution preparation step to form a heteropolyacid salt; and

a solvent evaporation step: which comprises evaporating a solvent from a mixture of a suspension containing aliphatic alcohols and a heteropolyacid salt catalyst prepared in the heteropolyacid salt formation step to isolate the catalyst as a solid.

The catalyst of the present invention is usually used in a reaction after calcining. The calcining temperature is usually 150° C. to 300° C., preferably 200° C. to 290° C. The calcining time is usually 1 hour to 10 hours, preferably 2 hours to 5 hours.

The heteropolyacid salt catalyst thus obtained is usually subjected to the pretreatment before it is used in an alkylation reaction of an aromatic compound, a transalkylation reaction or a disproportionation reaction of an aromatic compound or an alkyl aromatic compound, or an isomerization reaction of an alkyl-substituted aromatic compound. This is because it is important to dehydrate moisture contained in the catalyst. Since an alkylation reaction of an aromatic compound, a transalkylation reaction or a disproportionation reaction of an aromatic compound or an alkyl aromatic compound, or an isomerization reaction of an alkyl-substituted aromatic compound is a Friedel-Crafts reaction, the reaction does not proceed when the catalyst contains a large amount of moisture. In addition, when the pretreatment temperature is too high, the structure of a heteropolyacid is destructed, being not preferable. Examples of a method for the pre-treatment include a method comprising heating of a catalyst under a gas stream, a method comprising reduced-pressure drying of a catalyst under heating, and the like, but these methods are not particularly limited. For example, in a method comprising heating of a catalyst under a gas stream, examples of the gas include an inert gas, an air and the like. Important is the moisture content in the gas and a lower content is preferable. A nitrogen gas is preferably used. The pre-treatment temperature and time are the same values as the aforementioned calcining temperature and time. The temperature is important, and is preferably selected from 150° C. to 300° C. The pre-treatment time is usually 1 hour to 10 hours, preferably 2 hours to 5 hours. In the case of a method comprising reduced-pressure drying of a catalyst under heating, the heating temperature is preferably the same as that of the above-described stream method, and the pretreatment time is also preferably the same as that of the above-described stream method.

Hereinafter, an alkylating reaction of an aromatic compound, a transalkylation reaction or a disproportionation reaction of an aromatic compound and/or an alkyl aromatic compound, and an isomerization reaction of an alkyl-substituted aromatic compound using the catalyst of the present invention, which are the third to fifth aspects of the present invention, will be explained.

The catalyst of the present invention is effective in alkylation, transalkylation, disproportionation and isomerization of an aromatic compound, and is effective for various aromatic compounds and alkyl-substituted aromatic compounds such as benzene, monoalkylbenzene such as toluene, ethylbenzene and isopropylbenzene, polyalkylbenzene such as diethylbenzene and diisopropylbenzene, various alkylbenzenes, and other aromatic compounds, for example, naphthalene, indane, and tetralin. Examples of the aromatic compound also include compounds containing a heteroatom such as chlorobenzene and phenol. Examples of the aromatic compound also include poly-substituted aromatic compounds which are used in an isomerization reaction. A preferable aromatic compound is aromatic hydrocarbon, and examples of a produced alkyl aromatic compound include alkyl aromatic hydrocarbon. In addition, preferably, alkyl aromatic hydrocarbon substituted with 2 to 4 alkyl groups is used in an isomerization reaction. Further preferable examples include benzene and alkyl-substituted benzene.

Examples of olefin used in an alkylation reaction of an aromatic compound using the catalyst of the present invention, which is the third aspect of the present invention, include various olefins, such as linear alpha olefins or internal olefins such as ethylene, propylene, n-butene, isobutene, pentene and hexene; branched alpha olefins or internal olefins such as isopentene and isohexene; and cyclic olefins such as cyclohexene; preferably linear alpha olefins or internal olefins such as ethylene, propylene, n-butene, isobutene, pentene and hexene; further preferably linear alpha olefins having not more than 6 carbon atoms such as ethylene, propylene, n-butene, isobutene, pentene and hexene.

Examples of a starting material used in a transalkylation reaction or a disproportionation reaction of an aromatic compound using the catalyst of the present invention, which is the fourth aspect of the present invention, include benzene, ethylbenzene, cumene, diethylbenzene, diisopropylbenzene, triethylbenzene, triisopropylbenzene, tetraethylbenzene, tetraisopropylbenzene, polyethylbenzene, polyisopropylbenzene, and the like. Herein, polyalkylbenzene is a generic term for benzene having 2 or more alkyl substituents.

For example, a transalkylation reaction between benezene and diethybenzene can produce a mixture of benzene, ethylbenzene, diethybenzene and triethylbenzene. In addition, for example, a transalkylation reaction between benzene and diisopropylbenzene can produce a mixture of benzene, cumene, diisopropylbenzene and triisopropylbenzene. This is the same in the case of other alkyl groups.

For example, a disproportionation reaction of ethylbenzene can produce a mixture of benzene, ethylbenzene, diethylbenzene and triethylbenzene. In addition, for example, a disproportionation reaction of isopropylbenzene can produce a mixture of benzene, cumene, diisopropylbenzene and triisopropylbenzene. This is the same in the case of other alkyl groups.

Preferably, a transalkylation reaction or a disproportionation reaction for producing ethylbenzene, diethybenzene, cumene and diisopropylbenzene is performed.

Examples of a starting material used in an isomerization reaction of an aromatic compound using the catalyst of the present invention, which is the fifth aspect of the present invention, include o-diethybenzene, m-diethybenzene, p-diethybenzene and/or a mixture of compounds selected from these three kinds of compounds, and o-diisopropylbenzene, m-diisopropylbenzene, p-diisopropylbenzene and/or a mixture of compounds selected from these three kinds of compounds. Other groups substituted isomers may be also used, but aromatic compounds in which the aromatic nucleus is substituted with alkyl substituents are preferably used.

For example, an isomerization reaction of p-diethylbenzene using the catalyst of the present invention produces a mixture of o-diethybenzene and m-diethylbenzene, but at the same time, a transalkylation reaction and a disproportionation reaction proceed to produce a mixture of benzene, ethylbenzene and triethylbenzene. Similarly, an isomerization reaction of p-diisopropylbenzene using the catalyst of the present invention produces a mixture of o-diisopropylbenzene, m-diisopropylbenzene, p-diisopropylbenzene, benzene, cumene, and triisopropylbenzene.

In the reaction, preferably an aromatic compound in which the aromatic nucleus is substituted with 2 to 4 alkyl substituents, more preferably an aromatic compound in which benzene is substituted with two alkyl groups, particularly di-isopropylbenzene may be used.

Hereinafter, various reaction conditions in an alkylation reaction, a transalkylation reaction, a disproportionation reaction and an isomerization reaction of an aromatic compound using the catalyst of the present invention, which are the third to fifth aspects of the present invention, will be explained.

For example, when heteropolyacid leached out from the catalyst to reaction solution, it is problem that the process cannot be operated because the leached material blocks the distillation tower at the down stream of the reactor.

Then the purpose of this invention relates to producing a new heteropolyacid salt catalyst which doesn't leach out to a reaction solution. The amount of the heteropolyacid, which leaches out from the catalyst, is preferably 0 to 2 ppm by weight, more preferably 0 to 1 ppm by weight as a content in the reaction solution.

Since the activity of the catalyst is lost depending on the moisture content, management of an aromatic compound, olefin, an alkyl aromatic compound and a polyalkyl aromatic compound to be used is important. The moisture contained in an aromatic compound, olefin or an alkyl aromatic compound is preferably not more than 100 ppm as expressed by weight. Further preferably, the moisture is not more than 30 ppm. More preferably, the moisture is not more than 20 ppm.

Unless an aromatic compound contains a component causing oligomerization in the presence of an acid catalyst, impurities in an aromatic compound do not matter in use. Similarly, unless olefin contains a component causing oligomerization in the presence of an acid catalyst, impurities in olefin do not matter in use. A higher purity of an aromatic compound and olefin is preferable.

Examples of a reaction method include various reaction methods such as a fixed bed flow reaction system, a slurry flow reaction system and a batch reaction system, and an example of an industrially preferable reaction method is a fixed bed flow reaction system.

Examples of a method of reacting an aromatic compound, olefin or an alkyl aromatic compound in alkylation, transalkylation, disproportionation or isomerization include various reaction methods such as a method comprising supply of an aromatic compound, olefin or an alkyl aromatic compound in the liquid phase to the reaction, a method comprising injection of an olefin gas or an alkyl aromatic compound into an aromatic compound solution, and a method comprising a simultaneous reaction of an aromatic compound, olefin and an alkyl aromatic compound in the gas phase. Industrially preferable examples include a method comprising supply of an aromatic compound, olefin or an alkyl aromatic compound in the liquid state to the reaction, and a method comprising injection of an olefin gas or an alkyl aromatic compound into an aromatic compound solution.

In the case of a flow method, the supply amount of an aromatic compound, olefin or an alkyl aromatic compound relative to the catalyst amount is 0.1 to 200 h⁻¹ when expressed as LHSV (Liquid Hourly Space Velocity), on the basis of an aromatic compound. The mole ratio of aromatic compound/olefin or the mole ratio of aromatic compound/alkyl group is 1.0 to 5.0. When the mole ratio is high, a monoalkyl aromatic compound is produced in a large amount. When the mole ratio is low, a polyalkyl aromatic compound such as a dialkyl aromatic compound, a trialkyl aromatic compound or the like is produced in a large amount. The reaction temperature usually is 50° C. to 250° C., preferably 50° C. to 200° C. In the case of an alkylation reaction, the reaction temperature is preferably 50° C. to lower than 150° C. The reaction pressure is usually an atmospheric pressure to 10 MPa gauge, preferably 0.05 MPa gauge to 5 MPa gauge.

In the case of a batch reaction, the charging amount of an aromatic compound relative to the catalyst amount is usually 1.0 to 200 when expressed as weight ratio. The mole ratio of aromatic compound/olefin or the mole ratio of aromatic compound/alkyl group is usually between 1.0 and 5.0. The reaction temperature is usually 50° C. to 250° C., preferably 50° C. to 200° C. In the case of an alkylation reaction, the reaction temperature is preferably 50° C. to lower than 150° C. The reaction pressure is usually an atmospheric pressure to 10 MPa, preferably 0.05 MPa to 5 MPa. The reaction time is preferably 30 minutes to 5 hours.

The third aspect of the present invention is a process for producing an alkyl aromatic compound by alkylating an aromatic compound with olefin. Depending on the reaction conditions, in some cases, the ratio of an alkyl aromatic compound and a dialkyl aromatic compound which are simultaneously produced by said process may lean remarkably toward a dialkyl aromatic compound, and therefore an alkyl aromatic compound may be produced in a very small amount.

The catalyst of the present invention is particularly characteristic when the catalyst is supported on the above-described carrier. The catalyst supported on a carrier has pore structure which is governed by the pore structure of the carrier. This leads to production of larger amounts of a dialkyl aromatic compound and a trialkyl aromatic compound in an alkylation reaction of an aromatic compound, as compared with a zeolite catalyst having micropores which is normally used in producing an alkyl aromatic compound. The characteristic of the catalyst of the present invention is advantageous to industrial production of a dialkyl aromatic compound, and the catalyst of the present invention can produce a dialkyl aromatic compound at a rate such as can not be accomplished by using a zeolite catalyst having micropores. When a dialkyl aromatic compound is industrially used, the catalyst of the present invention is more preferable than a zeolite catalyst.

The fourth aspect of the present invention is transalkylation or disproportionation of an aromatic compound and/or an alkyl aromatic compound. In the fourth aspect, the catalyst of the present invention is influenced by the pore structure of a carrier when it is supported on the carrier, as in the case of alkylation. This leads to production of larger amounts of a dialkyl aromatic compound and a trialkyl aromatic compound in a transalkylation or disproportionation reaction of an aromatic compound and/or an alkyl aromatic compound, as compared with a zeolite catalyst having micropores which is normally used in producing an alkyl aromatic compound. The characteristic of the catalyst of the present invention is advantageous to industrial production of a dialkyl aromatic compound, and the catalyst of the present invention can produce a dialkyl aromatic compound at a rate such as can not be accomplished by using a zeolite catalyst having micropores. When a dialkyl aromatic compound is industrially used, the catalyst of the present invention is more preferable than a zeolite catalyst.

The fifth aspect of the present invention is an isomerization reaction for substitution positions of alkyl groups of an alkyl aromatic compound. In the fifth aspect, the characteristic of the catalyst of the present invention is influenced by the pore structure of a carrier when the catalyst is supported on the carrier, as in the case of alkylation. This leads to reactions of a m-isomer and a o-isomer similarly to a reaction of a p-isomer in an isomerization reaction, as compared with a zeolite catalyst having micropores which is normally used in producing an alkyl aromatic compound. The characteristic of the catalyst of the present invention is advantageous to industrial reactions of a m-isomer and an o-isomer, and the catalyst of the present invention can react a m-isomer and an o-isomer at a rate such as can not be accomplished by using a zeolite catalyst having micropores. When a m- or o-dialkyl aromatic compound is industrially reacted, the catalyst of the present invention is more preferable than a zeolite catalyst.

### EXAMPLES

### Example 1

### 1. Preparation of Catalyst

In 500 g of water was dissolved 990.54 g of commercially available 12-tungstosilicic acid (H₄SiW₁₂O₄₀, NIPPON INORGANIC COLOUR & CHEMICAL CO., LTD.). The aqueous solution was concentrated while heated to 45° C. with a rotary evaporator, to obtain a saturated aqueous solution, followed by recrystallization at 0° C. Crystals were suction-filtered to obtain 486.72 g of crystals. The mother liquor was heated again to 45° C., concentrated, and then recrystallized at 0° C. Crystals were suction-filtered to obtain 294.14 g of crystals. This procedure was repeated again to obtain 156.01 g of crystals. A total 936.87 g of the obtained crystals were ground and then air-dried to obtain crystal powder.

A silica carrier (Fuji Silysia Chemical Ltd, CARIACT-50) was sufficiently ground with a mortar and then calcined at 350° C. for 2 hours to obtain a silica carrier.

In 100 ml of dehydrated ethanol was dissolved 10.23 g of cesium hydroxide (manufactured by MP Biomedicals) using a messflask. When the solution of CsOH in ethanol was sampled using 10 ml of a whole pipette and then titrated with a 0.2 mol/l HCl standard solution (f=1.005), 29.15 ml of the HCl standard solution was needed. As a result, the solution of CsOH in ethanol was determined to be Cs⁺5.859×10⁻⁴ mol/l.

To 50 ml of dehydrated ethanol was added 5.0 g of the previously prepared 12-tungstosilicic acid, and the mixture was sufficiently stirred at room temperature to dissolve the material. To the solution was added 5.0 g of the silica carrier, followed by sufficient stirring. Ethanol was evaporated at 30° C. with a rotary evaporator to obtain 11.0 g of a white solid. The solid was dried in a drier at 70° C. to obtain 9.1 g of silica-supported 12-tungstosilicic acid. A calculated value of a supported amount was 50.0% by weight.

Into a mixed solvent of 25 ml of dehydrated ethanol and 25 ml of dehydrated heptane was dissolved 3.15 ml of the previously prepared solution of CsOH in ethanol using a whole pipette and a mess pipette. While the solution was sufficiently stirred, 9.1 g of the previously-prepared silica-supported 12-tungstosilicic acid was added, and the mixture was stirred for 2 minutes. Ethanol and heptane were evaporated from the obtained suspension with a rotary evaporator to obtain 9.32 g of a white solid. The solid was dried in a drier at 70° C. to obtain a silica-supported cesium 12-tungstosilicate partial salt catalyst. The chemical formula of the solid was determined to be 50% by weight of Cs_{1.37}H_{2.63}SiW₁₂O₄₀/SiO₂ on the basis of a calculated value.

### 2. Alkylation Reaction

The catalyst obtained as described above was molded into 1 to 2 mm, and 0.507 g of the catalyst together with 4.3 g of an α alumina sphere (1 mm) (Al₂O₃) as a diluent was put in a stainless reaction tube having an internal diameter of 10 mm and an external diameter of 12 mm.

A catalyst layer was heated to 250° C. for 2 hours under a nitrogen stream at 200 ml/min to calcine the catalyst. After cooled to room temperature, benzene and propylene were passed into the reaction tube at a predetermined pressure in an upflow manner while the catalyst layer was maintained at a predetermined temperature, to perform an alkylation reaction of benzene.

In the reaction tube, benzene was passed at 8.5 g/h under nitrogen, propylene was passed at 12.5 Nml/min, and the reaction pressure was maintained at 0.15 MPaG. The hot spot of the catalyst layer was 49.9° C.

After 6 hours from initiation of the reaction, the reaction solution was sampled and analyzed by gas chromatography. The propylene conversion was 42.0%, the cumene selectivity was 77.0%, and the diisopropylbenzene selectivity was 17.4% as a total value of three isomers.

### 3. Dissolution Test of Catalyst

The reaction solution was sampled at 3 hours to 8 hours after initiation of the reaction, brought into the form which could be analyzed by ICP emission analysis, and then subjected to microanalysis of tungsten (W) contained in the reaction solution. The W content in the reaction solution was not higher than a detection limit of 0.1 ppm, and was not higher than 0.13 ppm in terms of H₄SiW₁₂O₄₀.

### 4. Measurement of Acid Amount of Catalyst

The acid amount on the external surface of the catalyst was measured. First, 0.5 g of the catalyst was weighed, finely ground, and then heated to 250° C. for 2 hours under a nitrogen stream at 200 ml/min to calcine the catalyst. Then, the catalyst was transferred to a Schlenk flask, which was evacuated under vacuum at 130° C. while heated with an oil bath. Then, nitrogen was introduced into the Schlenk flask. In the Schlenk flask maintained at 100° C. under a nitrogen stream, a gas obtained by bubbling 2,6-dimethylpyridine (Kanto Chemical Co., Inc.; special grade; hereinafter, abbreviated as 2,6-DMPy) with another nitrogen stream was passed through the catalyst for 1 minute, and thereby 2,6-DMPy was adsorbed on the catalyst. Then, vacuum evacuation was performed at 100° C. for 1 hour. In order to subject the obtained catalyst to thermogravimetry (TG), 11.491 mg of a sample of the catalyst was placed on an apparatus (Regaku Thermo Plus TG 8120). The temperature of the sample was elevated at 10° C./min up to 1000° C., and measurement was performed. When the sample temperature was elevated up to 300° C., a decrease in the sample weight was 0.133 mg. When the sample temperature was elevated up to 900° C., decrease in the sample weight was 0.552 mg. The sample weight was reduced to 10.939 mg. In addition, 16.956 mg of a catalyst on which 2,6-DMPy was not absorbed was placed on the apparatus, and measurement was performed similarly. When the sample temperature was elevated up to 300° C., a decrease in the sample weight was 0.419 mg. When the sample temperature was elevated up to 900° C., a decrease in the sample weight was 0.565 mg. The sample weight was reduced to 16.391 mg. From the above results, the desorption amount of an adsorbed material desorbed from the catalyst on which 2,6-DMPy was adsorbed and the desorption amount of an adsorbed material desorbed from the catalyst on which 2,6-DMPy was not adsorbed could be calculated based on the weight of the catalyst. For the calculation, it was assumed that, in TG of the catalyst, adsorbed materials being physically adsorbed on the catalyst, for example water and the like, were desorbed during heating up to 300° C. In addition, it was assumed that a desorption amount from the catalyst during from 300° C. to 900° C. was the amount of OH present on the surface of a silica carrier. Further, it was assumed that all of a desorption amount from the catalyst on which 2,6-DMPy was adsorbed during from 300° C. to 900° C. was the amount of 2,6-DMPy together with OH present on the surface of a silica carrier. From these measured values, the acid amount on the external surface as described in claims was calculated. It was also thought that some 2,6-DMPy was adsorbed in micropores of the catalyst, but herein, a calculated value as obtained above was regarded as the acid amount on the external surface.

The desorption amount per the catalyst weight of an adsorbed material desorbed from the catalyst on which 2,6-DMPy was adsorbed was:

(0.552 mg−0.133 mg)/10.939 mg=0.419/10.939=38.30 mg/g-cat.

The desorption amount per the catalyst weight of an adsorbed material desorbed from the catalyst on which 2,6-DMPy was not adsorbed was:

(0.565 mg−0.419 mg)/16.391 mg=0.146/16.391=8.91 mg/g-cat.

The amount of 2,6-DMPy adsorbed on the catalyst was a difference between the above two measurements:

38.30-8.91=29.39 mg/g-cat.

Since the molecular weight of 2,6-DMPy is 107.15, the acid amount of the catalyst was:

29.39 mg/g-cat/107.15=0.274 mmol/g-cat=274 μmol/g-cat.

Since the content of a cesium 12-tungstosilicate partial salt was 50% by weight, the acid amount per heteropolyacid salt (hereinafter, abbreviated as HPA) was:

274 μmol/g-cat/0.5=549 μmol/g-HPA.

### 5. Electron Microscope Observation of Catalyst

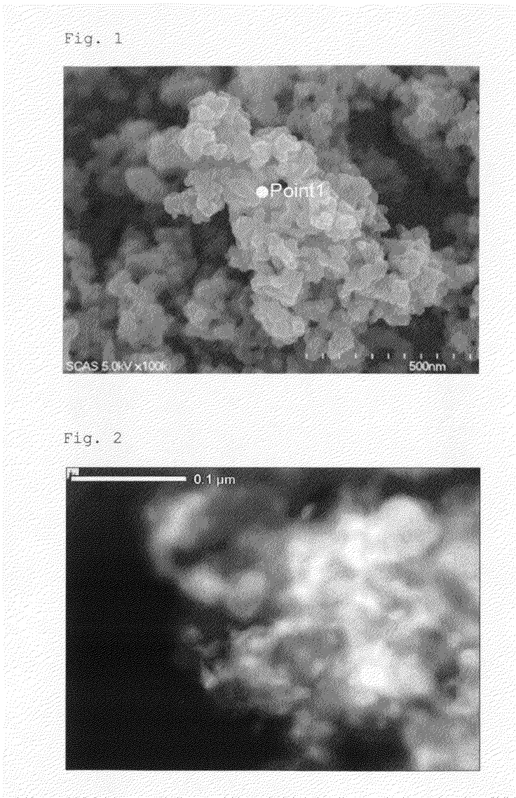

Then, the catalyst (50% by weight Cs_{1.37}H_{2.63}SiW₁₂O₄₀/SiO₂) was observed with a scanning electron microscope (hereinafter, abbreviated as SEM; HITACHI FE-SEM S-4700) at a 100,000 magnification. A result is shown in FIG. 1. The measurement condition was an acceleration voltage of 5 kV. As seen from FIG. 1, the cesium 12-tungstosilicate partial salt consisted of crystal particles having an average particle diameter in the short axis of 42.2 nm. 25 particles are measured. In the catalyst, a result of SEM observation did not show a larger particle having a larger particle diameter than the average particle diameter. Thus, the proportion of particles having this average particle diameter in the total particles was approximately not less than 90%.

As seen from spectra (FIG. 8) of energy dispersive X-ray spectrometer (EDX) of this crystal particle, a Cs element and a W element were detected. Thus, the crystal particle was found to be a cesium 12-tungstosilicate partial salt.

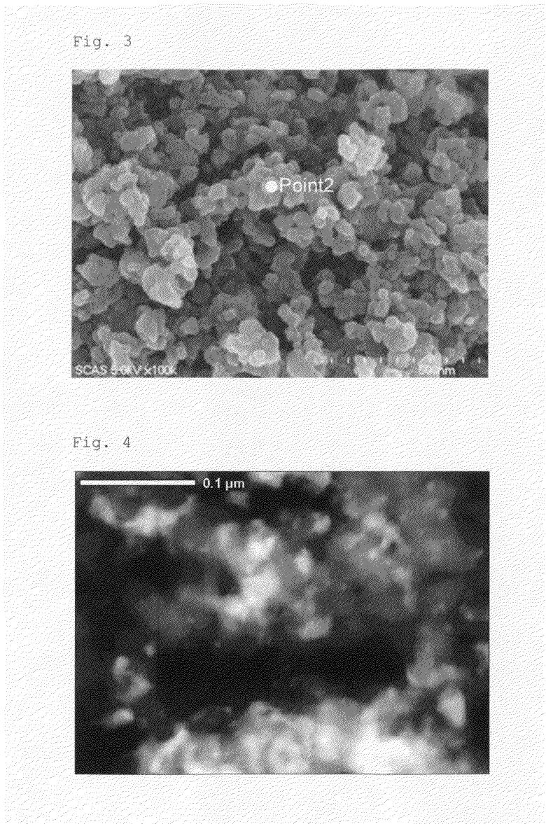

Further, the catalyst was observed by field-emission scanning transmission electron microscopy (we abbreviate field-emission scanning transmission electron microscope to FE-STEM) at a 500,000 magnification. In a dark field image observed by this technique (FIG. 2), unlike a commonly observed bright field image by transmission electron microscopy, heavier elements are seen brighter. Apparatuses and conditions used in the observation are as follows. The apparatuses were JEM-2200FS field-emission transmission electron microscope (FE-TEM) equipped with scanning transmission electron microscope (STEM) system and JED-2300T energy dispersive X-ray spectrometer (EDX) both manufactured by JEOL. Ltd. The conditions were an acceleration voltage of 200 kV, a beam diameter of 1.5 nm, a camera length of 4 cm, and a sample tilt angle of 15°. As a result of observation, it was seen that a heteropolyacid salt particle supported on a SiO₂ particle showed an image with brighter regions in which heavy elements such as tungsten and cesium were uniformly supported on SiO₂. Further, in the elemental map which was obtained simultaneously with the observation, Si, O, Cs and W were distributed approximately similarly. Also from the result, it was seen that the cesium 12-tungstosilicate partial salt was uniformly supported on SiO₂.

### Example 2

### 1. Preparation of Catalyst

A silica carrier (Fuji Silysia Chemical Ltd, CARIACT-50) was sufficiently ground with a mortar, and then calcined at 350° C. for 2 hours to obtain a silica carrier.

Into a mixed solvent of 100 ml of dehydrated ethanol and 100 ml of heptane was dissolved 5.02 g of 12-tungstosilicic acid prepared in Example 1. Into the solution was suspended 5.0 g of the silica carrier, while sufficiently stirred. To the suspension was added dropwise 6.26 ml of cesium hydroxide prepared in Example 1 over 16 minutes, while sufficiently stirred. After completion of addition dropwise, the mixture was stirred for 17 minutes. Then, a solvent from the resulting white suspension was removed using a rotary evaporator at 30° C. to 50° C. to obtain 10.0 g of a white solid. The chemical formula of the solid was determined to be 50% by weight of Cs_{2.5}H₁₅SiW₁₂O₄₀/SiO₂ on the basis of a calculated value. The white solid was dried well in a dryer at 70° C. to obtain 9.9 g of a white solid. Then, in order to wash off the remaining heptane, 100 ml of dehydrated hexane was added to the solid, this was sufficiently stirred, a supernatant was discarded, and the remaining hexane was removed using a rotary evaporator at 30° C. to 50° C. to obtain 9.8 g of a white solid. The solid was sufficiently dried in a dryer at 70° C. to obtain 9.7 g of a silica-supported cesium 12-tungstosilicate partial salt catalyst.

### 2. Alkylation Reaction

The catalyst obtained as described above was molded into 1 to 2 mm, and an alkylation reaction of benzene was performed by the method shown in Example 1.

In the alkylation reaction, 0.51 g of the catalyst was used, the flow rate of benzene was 9.7 g/h, the flow rate of propylene was 12.5 Nml/min, the reaction pressure was 0.15 MPa, and the hot spot of a catalyst layer was 50.0° C.

After 6 hours from initiation of the reaction, the reaction solution was sampled and analyzed by gas chromatography. The propylene conversion was 13.0%, the cumene selectivity was 88.9%, and the diisopropylbenzene selectivity was 8.16% as a total of three isomers.

### 3. Measurement of Acid Amount of Catalyst

According to the same manner as that of Example 1, the acid amount on the external surface of the catalyst was measured. Results are shown below.

A sample (15.853 mg) on which 2,6-DMPy was adsorbed was subjected to TG.

Further, a catalyst (16.577 mg) on which 2,6-DMPy was not adsorbed was subjected to TG.

The desorption amount per the catalyst weight of an adsorbed material desorbed from the catalyst on which 2,6-DMPy was adsorbed was:

(0.544 mg−0.173 mg)/15.294 mg=0.371/15.294=24.26 mg/g-cat.

The desorption amount per the catalyst weight of an adsorbed material desorbed from the catalyst on which 2,6-DMPy was not adsorbed was:

(0.568 mg−0.414 mg)/16.009 mg=0.154/16.009=9.62 mg/g-cat.

The amount of 2,6-DMPy adsorbed on the catalyst was a difference between the above two measurements:

24.26−9.62=14.64 mg/g-cat.

Since the molecular weight of 2,6-DMPy is 107.15, the acid amount of the catalyst was:

14.64 mg/g-cat/107.15=0.137 mmol/g-cat=137 μmol/g-cat.

Since the content of a cesium 12-tungstosilicate partial salt was 50% by weight, the acid amount per heteropolyacid salt (HPA) was:

137 μmol/g-cat/0.5=273 μmol/g-HPA.

### 4. Electron Microscope Observation of Catalyst

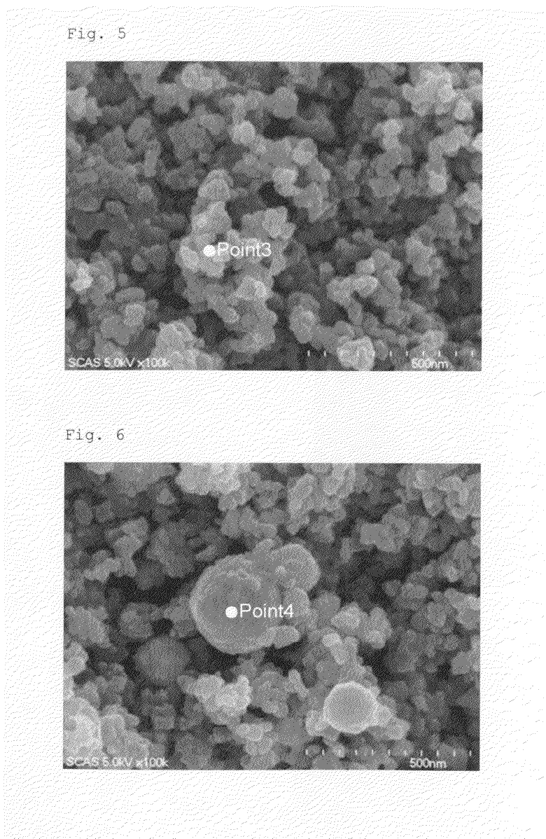

The catalyst (50% by weight of Cs_{2.5}H_{1.5}SiW₁₂O₄₀/SiO₂) was observed with a scanning electron microscope (SEM) as in Example 1. A result is shown in FIG. 3. As seen from FIG. 3, the cesium 12-tungstosilicate partial salt consisted of crystal particles having an average particle diameter in the short axis of 46.9 nm. 22 particles are measured. In the catalyst, a result of SEM observation did not show a larger particle having a larger particle diameter than the average particle diameter. Thus, the proportion of particles having this average particle diameter in the total particles was approximately not less than 90%.

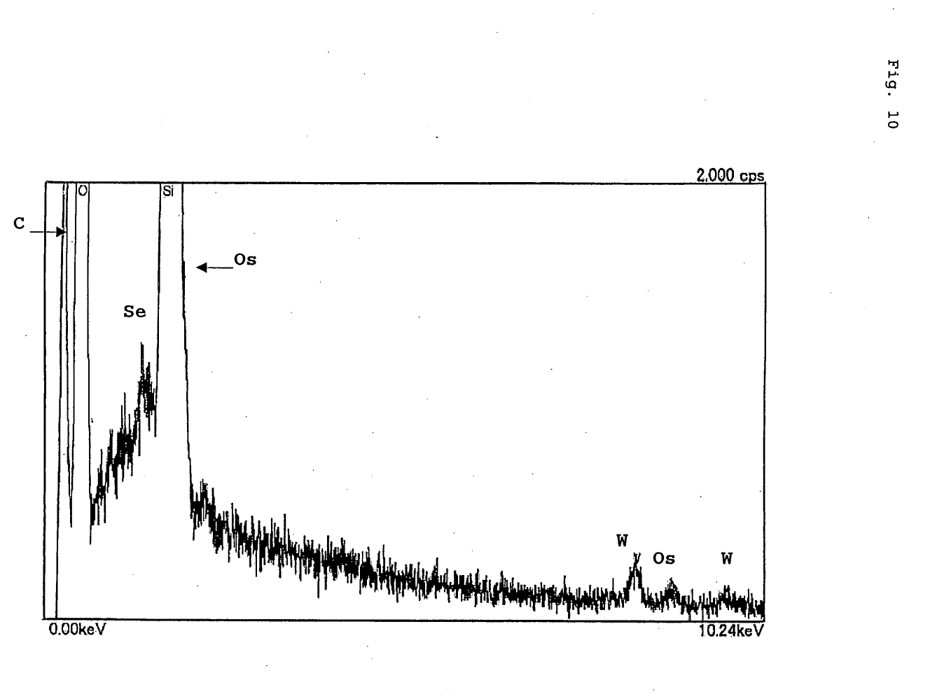

As seen from spectra (FIG. 9) of energy dispersive X-ray spectrometer (EDX) of this crystal particle, a Cs element and a W element were detected. Thus, the crystal particle was found to be a cesium 12-tungstosilicate partial salt.

Further, the catalyst was observed with field-emission scanning transmission electron microscopy (FE-STEM) at a 500,000 magnification as in Example 1. In an observed dark field image (FIG. 4), unlike a commonly observed bright field image by transmission electron microscopy, heavier elements are seen brighter. As a result of observation, it was seen that a heteropolyacid salt particle supported on a SiO₂ particle showed an image with brighter regions in which heavy elements such as tungsten and cesium were uniformly supported on SiO₂. Further, in the elemental map which was obtained simultaneously with the observation, Si, O, Cs and W were distributed approximately similarly. Also from the result, it was seen that the cesium 12-tungstosilicate partial salt was uniformly supported on SiO₂

### Example 3

### 1. Preparation of Catalyst

12-Tungstosilicic acid which was prepared in the example 1 was used.

A silica carrier (Fuji Silysia Chemical Ltd, CARIACT-50) was sufficiently ground with a mortar and then calcined at 350° C. for 2 hours to obtain a silica carrier.

In 100 ml of dehydrated ethanol was dissolved 14.29 g of cesium hydroxide (manufactured by MP Biomedicals) using a messflask. When the solution of CsOH in ethanol was sampled using 10 ml of a whole pipette and then titrated with a 0.2 mol/l HCl standard solution (f=1.005), 40.9 ml of the HCl standard solution was needed. As a result, the solution of CsOH in ethanol was determined to be Cs⁺8.221×10⁻⁴ mol/l.

To 50 ml of dehydrated ethanol was added 5.0 g of the previously prepared 12-tungstosilicic acid, and the mixture was sufficiently stirred at room temperature to dissolve the material. To the solution was added 5.0 g of the silica carrier, followed by sufficient stirring. Ethanol was evaporated at 25° C. with a rotary evaporator to obtain 11.4 g of a white solid. The solid was dried in a drier at 70° C. to obtain 9.4 g of silica-supported 12-tungstosilicic acid. A calculated value of a supported amount was 50.0% by weight.

Into a mixed solvent of 25 ml of dehydrated ethanol and 25 ml of dehydrated hexane was dissolved 2.23 ml of the previously prepared solution of CsOH in ethanol using a mess pipette. While the solution was sufficiently stirred, 9.4 g of the previously prepared silica-supported 12-tungstosilicic acid was added, and the mixture was stirred for 2 minutes. Ethanol and hexane were evaporated from the obtained suspension with a rotary evaporator to obtain 11.9 g of a white solid. The solid was dried in a drier at 70° C. to obtain 9.4 g of a silica-supported cesium 12-tungstosilicate partial salt catalyst. The chemical formula of the solid was determined to be 50% by weight of Cs_{1.30}H_{2.70}SiW₁₂O₄₀/SiO₂ on the basis of a calculated value.

### 2. Isomerization Disproportionation Transalkylation

The catalyst obtained as described above was molded into 1 to 2 mm, and 5.0 g of the catalyst was put in a stainless reaction tube having an internal diameter of 10 mm and an external diameter of 12 mm.

A catalyst layer was heated to 250° C. for 2 hours under a nitrogen stream at 200 ml/min to calcine the catalyst. After cooled to room temperature, dialkylbenzenes which contains the components as follows were passed into the reaction tube at a predetermined pressure in an upflow manner while the catalyst layer was maintained at a predetermined temperature, to perform a reaction. the reactant contains 97.15% of p-diisopropylbenzene (hereinafter, denoted PDB), 2.17% of o-diisopropylbenzene (hereinafter, denoted ODB), 0.16% of m-diisopropylbenzene (hereinafter, denoted MDB), 0.52% of the others.

In the reaction tube, the dialkylbenzenes was passed at 11.9 g/h under nitrogen, and the reaction pressure was maintained at 0.15 MPaG. The hot spot of the catalyst layer was 110° C.

After 22 hours from initiation of the reaction, the reaction solution was sampled and analyzed by gas chromatography. The reaction solution contains 5.59% of cumene, 16.02% of MDB, 0.14% of ODB, 68.46% of PDB, 9.15% of triisopropylbenzene, 0.64% of the others. These data shows that the conversion of ODB was 93.5%.

### Reference Example 1

### 1. Preparation of Catalyst

A silica carrier (Fuji Silysia Chemical Ltd, CARIACT-50) was sufficiently ground with a mortar, and then calcined at 350° C. for 2 hours to obtain a silica carrier.

To 100 ml of dehydrated ethanol was added 10.0 g of 12-tungstosilicic acid prepared in Example 1, and the mixture was sufficiently stirred at room temperature. To the mixture was added 10.0 g of the silica carrier, and then stirred. Ethanol was evaporated with a rotary evaporator at 30° C. to obtain 22.0 g of a white solid. The solid was dried in a dryer at 70° C. to obtain 18.8 g of silica-supported 2-tungstosilicic acid. A calculated value of a supported amount was 50.0% by weight.

Into 50 ml of distilled water was dissolved 3.15 ml of the solution of CsOH in ethanol prepared in Example 1 using a whole pipette and a mess pipette. While the solution was sufficiently stirred, 9.4 g of the previously prepared silica-supported 12-tungstosilicic acid was added, and the mixture was stirred for 5 minutes. Water and ethanol were evaporated from the obtained suspension with a rotary evaporator to obtain 9.74 g of a white solid. The solid was dried in a drier at 70° C. to obtain a silica-supported cesium 12-tungstosilicate partial salt catalyst. The chemical formula of the solid was determined to be 50% by weight of Cs_{1.33}H_{2.67}SiW₁₂O₄₀/SiO₂ on the basis of a calculated value.

### 2. Electron Microscope Observation of Catalyst

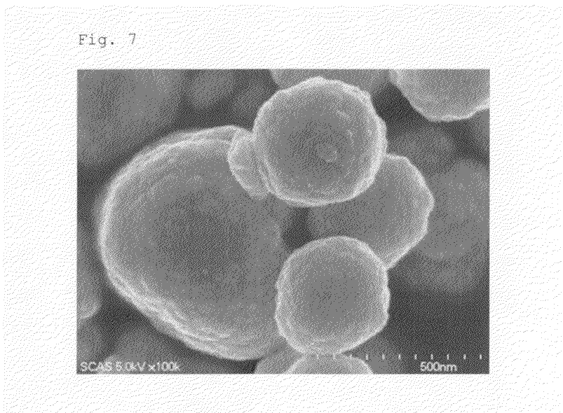

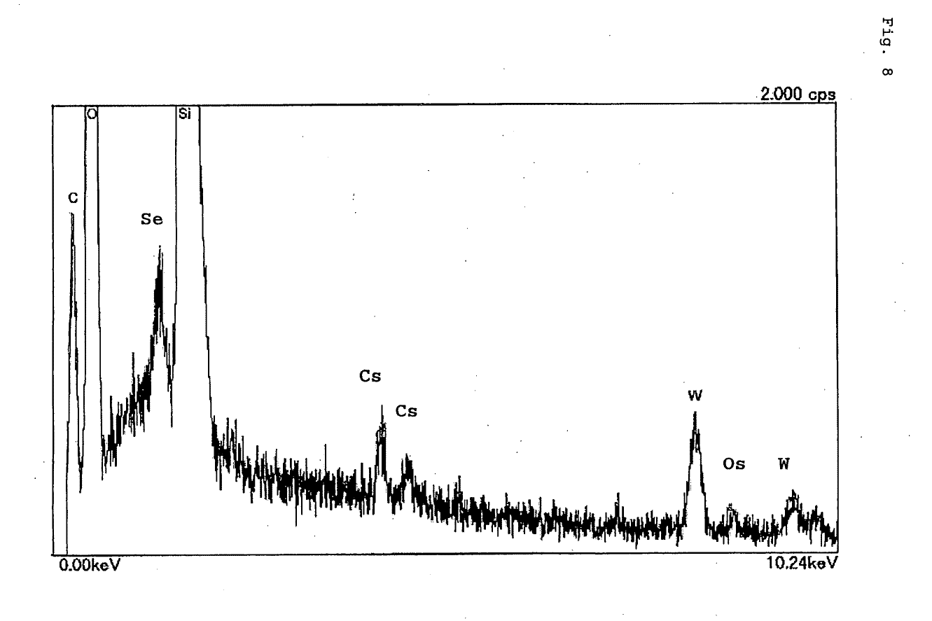

The catalyst (50% by weight of Cs_{1.33}H_{2.67}SiW₁₂O₄₀/SiO₂) was observed with a scanning electron microscope as in Example 1. A result is shown in FIG. 5. As seen from FIG. 5, the catalyst consisted of crystal particles having an average particle diameter in the short axis of 47.5 nm. 35 particles are measured. However, as shown in FIG. 6, there were rarely particles having a particle diameter in the short axis direction of not less than 300 nm.

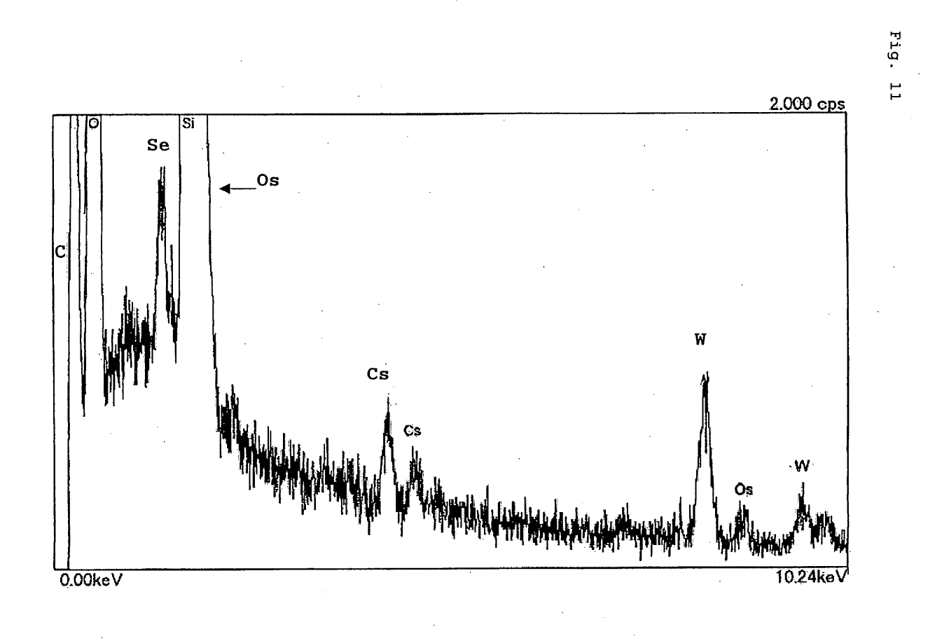

As seen from spectra of energy dispersive X-ray spectrometer (EDX) of this crystal particle, a Cs element was not detected and a W element was detected from EDX (FIG. 10) corresponding to FIG. 5. Thus, the crystal particle was found to be not a cesium 12-tungstosilicate partial salt. In addition, a Cs element and a W element were detected from EDX (FIG. 11) corresponding to FIG. 6. Thus, the crystal particle having a particle diameter in the short axis of not less than 300 nm was found to be a cesium 12-tungstosilicate partial salt.

### Comparative Example 1

### 1. Preparation of Catalyst

A silica carrier (Fuji Silysia Chemical Ltd, CARIACT-10) was sufficiently ground with a mortar, 21.0 g of which was calcined at 350° C. for 2 hours to obtain a silica carrier.

To 150 ml of distilled water was added 4.6 g of 12-tungstosilicic acid prepared in Example 1, and the mixture was sufficiently stirred at room temperature. To the mixture was added 10.0 g of the silica carrier, and then stirred sufficiently. Water was evaporated with a rotary evaporator at 50° C. to obtain 22.35 g of a white solid. The solid was dried in a dryer at 70° C. to obtain 14.3 g of silica-supported 2-tungstosilicic acid. A calculated value of a supported amount was 28.6% by weight as a value excluding crystallization water.

### 2. Alkylation Reaction

The catalyst obtained as described above was molded into 1 to 2 mm, and an alkylation reaction of benzene was performed by the method shown in Example 1.

In the alkylation reaction, 0.50 g of the catalyst was used, the flow rate of benzene was 10.5 g/h, the flow rate of propylene was 12.5 Nml/min, the reaction pressure was 0.15 MPa, and the hot spot of a catalyst layer was 53.0° C.

### 3. Leaching Test of Catalyst

The reaction solution was sampled at 22.5 hours to 25.5 hours after initiation of the reaction, brought into the form which could be analyzed by ICP emission analysis, and then subjected to microanalysis of tungsten (W) contained in the reaction solution. The W content in the reaction solution 1.7 ppm, and was 2.2 ppm in terms of H₄SiW₁₂O₄0. A detection limit was 0.1 ppm in terms of W.

### Comparative Example 2

### 1. Preparation of Catalyst

A silica carrier (Fuji Silysia Chemical Ltd, CARIACT G-10) was sufficiently ground with a mortar, 15.4 g of which was calcined at 350° C. for 2 hours to obtain 14.7 g of a silica carrier.

Cesium carbonate (Nacalai tesque, Inc.; guaranteed) (6.40 g) was calcinated at 450° C. for 2 hours in nitrogen to obtain 6.297 g of anhydrous cesium carbonate. This was dissolved in distilled water using a 100 ml messflask to prepare a Cs⁺3.865×10⁻⁴ mol/l aqueous solution.

12-Tungstosilicic acid (18.29 g) prepared in Example 1 was dissolved in 100 ml of distilled water, and 35.4 ml of the previously prepared cesium carbonate was added dropwise over 21 minutes to the solution while sufficiently stirred. After completion of addition dropwise, the mixture was sufficiently stirred, and allowed to stand overnight. Then, water was removed from the resulting white suspension at 40° C. using a rotary evaporator to obtain 18.61 g of a white solid. The chemical formula of the solid was determined to be Cs_{2.5}H_{1.5}SiW₁₂O₄₀ on the basis of a calculated value. The white solid was sufficiently dried in a dryer at 70° C.

The resulting Cs_{2.5}H_{1.5}SiW₁₂O₄₀ (1.0 g) and 1.52 g of a silica carrier were suspended in 50 ml of distilled water, the suspension was sufficiently stirred, and water was removed from the resulting white suspension at 40° C. using a rotary evaporator to obtain 2.55 g of a white solid. Further, the white solid was sufficiently dried in a dryer at 70° C. to obtain 2.53 g of a silica-supported cesium 12-tungustosilicate partial salt catalyst (40% Cs_{2.5}H_{1.5}SiW₁₂O₄₀/SiO₂).

### 2. Alkylation Reaction

The catalyst obtained as described above was molded into 1 to 2 mm, and an alkylation reaction of benzene was performed by the method shown in Example 1.

In the alkylation reaction, 0.50 g of the catalyst was used, the flow rate of benzene was 10.9 g/h, the flow rate of propylene was 12.5 Nml/min, the reaction pressure was 0.15 MPa, and the hot spot of a catalyst layer was 49.7° C.

After 6 hours from initiation of the reaction, the reaction solution was sampled and analyzed by gas chromatography. The propylene conversion was 11.8%, the cumene selectivity was 89.4%, and the diisopropylbenzene selectivity was 3.79% as a total of three isomers.

### 3. Electron Microscope Observation of Catalyst

In order to observe the shape of a cesium 12-tungustosilicate partial salt with an electron microscope, Cs_{2.5}H_{1.5}SiW₁₂O₄₀ was prepared similarly.

Cesium carbonate (9.75 g) was calcined similarly to obtain 9.634 g of anhydrous cesium carbonate. The anhydrous cesium carbonate was dissolved in water to prepare a Cs⁺5.913×10⁻⁴ mol/l aqueous solution.

12-Tungustosilicic acid (9.09 g) prepared in Example 1 was dissolved in 50 ml of distilled water, and 11.5 ml of cesium carbonate was added to the solution while sufficiently stirred. After completion of addition dropwise, the mixture was sufficiently stirred, and allowed to stand overnight. Then, water was removed from the resulting white suspension at 40° C. using a rotary evaporator to obtain a white solid. The chemical formula of the solid was determined to be Cs_{2.5}H_{1.5}SiW₁₂O₄₀ on the basis of a calculated value. The white solid was sufficiently dried in a dryer at 70° C.

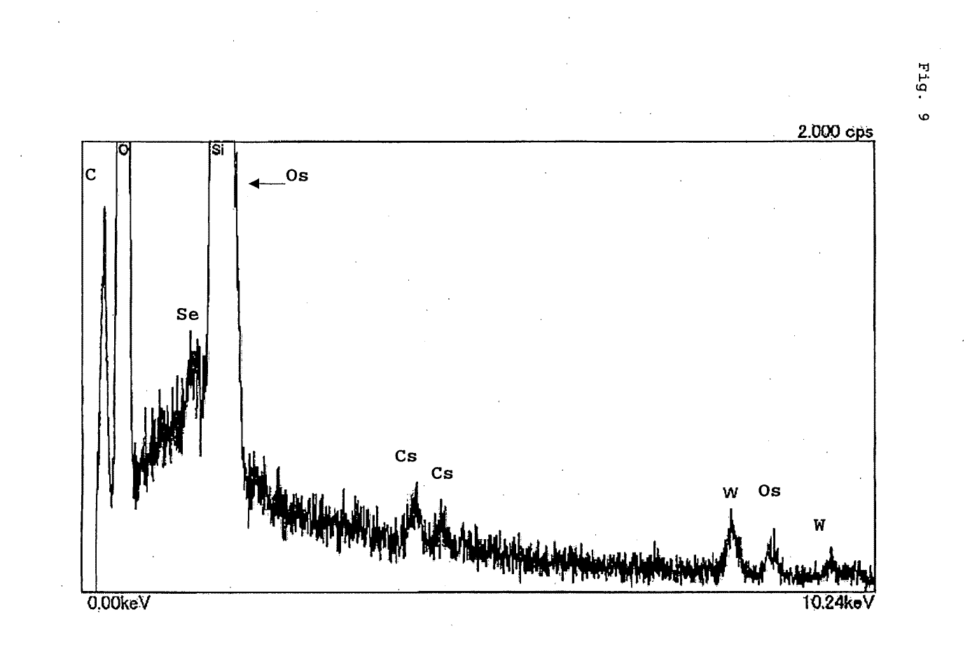

The white solid (Cs_{2.5}H_{1.5}SiW₁₂O₄₀) was observed with a scanning electron microscope (SEM) as in Example 1. A result is shown in FIG. 7. As seen from FIG. 7, the cesium 12-tungustosilicate partial salt consisted of crystal particles having an average particle diameter in the short axis of 430 nm. 4 particles are measured. In the catalyst, a result of SEM observation did not show a larger particle having a larger particle diameter than the average particle diameter. Thus, the proportion of particles having this average particle diameter in the total particles was approximately not less than 90%.

### 4. Measurement of Acid Amount of Catalyst

### Preparation of Catalyst

The white solid (Cs_{2.5}H_{1.5}SiW₁₂O₄₀) (1.00 g) used in electron microscope observation of a catalyst and a silica carrier (Fuji Silysia Chemical Ltd, CARIACT G-10) were sufficiently ground with a mortar, and calcined at 350° C. for 2 hours, 1.00 g of which was suspended in 50 ml of water and sufficiently stirred. The suspension was then heated with a rotary evaporator to evaporate water, to obtain 2.0 g of a white solid (50% Cs_{2.5}H_{1.5}SiW₁₂O₄₀/SiO₂). Then, the solid was sufficiently dried in a dryer at 70° C.

According to the same manner as that of Example 1, the acid amount on the external surface of the catalyst was measured. Results are shown below.

A sample (19.319 mg) on which 2,6-DMPy was adsorbed was subjected to TG.

Further, 12.549 mg of a catalyst on which 2,6-DMPy was not adsorbed was subjected to TG.

The desorption amount per the catalyst weight of an adsorbed material desorbed from the catalyst on which 2,6-DMPy was adsorbed was:

(0.561 mg−0.193 mg)/18.758 mg=0.368/18.758=19.62 mg/g-cat.

The desorption amount per the catalyst weight of an adsorbed material desorbed from the catalyst on which 2,6-DMPy was not adsorbed was:

(0.429 mg−0.305 mg)/12.120 mg=0.124/12.120=10.23 mg/g-cat.

The amount of 2,6-DMPy adsorbed on the catalyst was a difference between the above two measurements:

19.62-10.23=9.39 mg/g-cat.

Since the molecular weight of 2,6-DMPy is 107.15, the acid amount of the catalyst was:

9.39 mg/g-cat/107.15=0.0876 mmol/g-cat=87.6 μmol/g-cat.

Since the content of the cesium 12-tungustosilicate partial salt was 50% by weight, the acid amount per heteropolyacid salt (HPA) was:

87.6 μmol/g-cat/0.5=175 μmol/g-HPA.

### Comparative Example 3

### 1. Catalyst

Mordenite (Tosoh HSZ-690HOD1A, SiO₂/Al2O3=230, 1.5 mm extruded) was used.

### 2. Isomerization Disproportionation Transalkylation

5.0 g of the catalyst was put in a stainless reaction tube having an internal diameter of 10 mm and an external diameter of 12 mm.

A catalyst layer was heated to 250° C. for 2 hours under a nitrogen stream at 200 ml/min to calcine the catalyst. After cooled to room temperature, dialkylbenzenes which is as same as example 3 were passed into the reaction tube at a predetermined pressure in an upflow manner while the catalyst layer was maintained at a predetermined temperature, to perform a reaction as described at the example 3.

In the reaction tube, the dialkylbenzenes was passed at 12.2 g/h under nitrogen, and the reaction pressure was maintained at 0.15 MPaG. The hot spot of the catalyst layer was 170° C.

After 16 hours from initiation of the reaction, the reaction solution was sampled and analyzed by gas chromatography. The reaction solution contains 0.78% of cumene, 17.54% of MDB, 2.12% of ODB, 78.33% of PDB, 0.48% of triisopropylbenzene, 0.75% of the others. These data shows that the conversion of ODB was 2.3%.

1. A heteropolyacid salt catalyst for use in an alkylation reaction of an aromatic compound or a transalkylation, disproportionation or isomerization reaction of an alkyl aromatic compound, which comprises a heteropolyacid salt catalyst represented by the following formula (1):
H₄₋ₘZₘSiXₗ₂O₄₀ (1)
wherein X represents W or Mo, Z represents (NH₄) or an alkali metal atom, and m represents a numerical value of 0≦m≦4, and comprising a heteropolyacid salt crystal having an average particle diameter in the short axis of the crystal of less than 300 nm as a main component, wherein said heteropolyacid salt catalyst has an acid amount on the external surface of not less than 190 μmol per weight of a heteropolyacid salt.

2. The heteropolyacid salt catalyst according to claim 1, wherein the acid amount on the external surface of the catalyst is not less than 280 μmol per weight of a heteropolyacid salt.

3. A process for producing the heteropolyacid salt catalyst according to claim 1, which comprises preparing a heteropolyacid represented by the following formula (2):
H₄SiX₁₂O₄₀ (2)
wherein X represents W or Mo, or the heteropolyacid supported on a carrier by salt formation from a solution of an ammonium or alkali metal compound in the presence of an aliphatic alcohol solvent or aliphatic alcohols containing an organic solvent and/or a water solvent.

4. The process for producing a heteropolyacid salt catalyst according to claim 3, wherein the salt formation comprises:
a heteropolyacid dissolving step which comprises dissolving a heteropolyacid represented by the following formula (2):
H₄SiX₁₂O₄₀ (2)
wherein X represents W or Mo, in a saturated aliphatic hydrocarbon organic solvent containing aliphatic alcohols;
an alkali solution preparation step which comprises dissolving an alkali metal compound in an aliphatic alcohol;
a heteropolyacid salt formation step which comprises adding a solution prepared in the alkali solution preparation step to a heteropolyacid solution prepared in the heteropolyacid dissolving step to form a heteropolyacid salt; and
a solvent evaporation step which comprises evaporating a solvent from a mixture of a solution containing aliphatic alcohols and a heteropolyacid salt catalyst prepared in the heteropolyacid salt formation step to isolate the catalyst as a solid.

5. The process for producing a heteropolyacid salt catalyst according to claim 3, wherein the salt formation comprises:
a heteropolyacid support step which comprises supporting a heteropolyacid represented by the following formula (2):
H₄SiX₁₂O₄₀ (2)
wherein X represents W or Mo, on a carrier to prepare a supported heteropolyacid;
an alkali solution preparation step which comprises dissolving an ammonium or alkali metal compound in an aliphatic alcohol solvent or aliphatic alcohols containing an organic solvent and/or a water solvent;
a heteropolyacid salt formation step which comprises adding the supported heteropolyacid to a solution prepared in the alkali solution preparation step to form a heteropolyacid salt; and
a solvent evaporation step which comprises evaporating a solvent from a mixture of a solution containing aliphatic alcohols and a heteropolyacid salt catalyst prepared in the heteropolyacid salt formation step to isolate the catalyst as a solid.

6. A process for producing an alkyl aromatic compound by alkylation, which comprises contacting an aromatic compound with olefin in the presence of the heteropolyacid salt catalyst according to claim 1 or 2.

7. A process for producing an alkyl aromatic compound by alkylation comprising contacting an aromatic compound with olefin in the presence of the heteropolyacid salt catalyst according to claim 1 or 2, wherein a heteropolyacid leached out at a concentration of not more than 2 ppm by weight in a reaction solution.

8. A process for producing an alkyl aromatic compound by a transalkylation reaction or a disproportionation reaction, which comprises contacting an aromatic compound and/or an alkyl aromatic compound with a polyalkyl aromatic compound in the presence of the heteropolyacid salt catalyst according to claim 1 or 2.

9. A process for producing an alkyl aromatic compound by a transalkylation reaction or a disproportionation reaction comprising contacting an aromatic compound and/or an alkyl aromatic compound with a polyalkyl aromatic compound in the presence of the heteropolyacid salt catalyst according to claim 1 or 2, wherein a heteropolyacid leached out at a concentration of not more than 2 ppm by weight in a reaction solution.

10. A process for producing a di or more-substituted alkyl aromatic compound, which comprises performing an isomerization reaction for substitution positions of alkyl groups of a di or more-substituted alkyl aromatic compound in the presence of the heteropolyacid salt catalyst according to claim 1 or 2.

11. A process for producing a di or more-substituted alkyl aromatic compound comprising performing an isomerization reaction for substitution positions of alkyl groups of a di or more-substituted alkyl aromatic compound in the presence of the heteropolyacid salt catalyst according to claim 1 or 2, wherein a heteropolyacid leached out at a concentration of not more than 2 ppm by weight in a reaction solution.

12. A process for producing cumene and/or diisopropylbenzene, which comprises contacting benzene with propylene in the presence of the heteropolyacid salt catalyst according to claim 1 or 2.

13. A process for producing cumene and/or diisopropylbenzene comprising contacting benzene with propylene in the presence of the heteropolyacid salt catalyst according to claim 1 or 2, wherein a heteropolyacid leached out at a concentration of not more than 2 ppm by weight in a reaction solution.
